(19) United States
(12) Patent Application Publication
(10) Pub. No.: US 2004/0202621 A1
(43) Pub. Date: Oct. 14, 2004

(54) Teeth whitening system based on the combination of hydrogen peroxide and carbamide peroxide

(76) Inventors: Jan A. Orlowski, Alta Dena, CA (US); David V. Butler, West Covina, CA (US); Thomas Shiah, Chino Hills, CA (US); Mahin Mapar, Torrance, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/746,124

(22) Filed: Dec. 23, 2003

Related U.S. Application Data
(60) Provisional application No. 60/436,118, filed on Dec. 23, 2002.

(51) Int. Cl. A61K 7/20

(52) U.S. Cl. 424/53

(57) ABSTRACT

Disclosed are compositions for bleaching teeth, comprising a single component part or two or more components blended together before each application. The compositions offer extended shelf life and accelerated bleaching action while significantly reducing the possibility of user discomfort. In preferred embodiments, the composition or one of its parts comprises a blend of hydrogen peroxide and carbamide peroxide in a solution having the consistency of a gel or a viscous liquid. The inventive system allows for water in the composition to avoid tissue desiccation and associated user discomfort, without compromising the shelf life of the composition.

### RELATED APPLICATION INFORMATION

This application claims priority under 35 U.S.C. § 119(e) to U.S. Provisional Patent Application No. 60/436,118, filed Dec. 23, 2002, the disclosure of which is hereby incorporated by reference in its entirety.

### BACKGROUND OF THE INVENTION

1. Field of the Invention

This invention relates generally to teeth whiteners, specifically to teeth whitening featuring increased storage stability while offering superior bleaching efficacy.

### BACKGROUND OF THE INVENTION

Teeth whiteners, also known as teeth bleaching agents, are in widespread use as a cosmetic means to enhance appearance, while also contributing to better oral health and hygiene. Particularly popular and effective among these agents are those whose chemistry is based on peroxides, of which hydrogen peroxide and carbamide peroxide (an adduct of hydrogen peroxide and urea) are most frequently employed. Such peroxides are characterized by their ability to generate radical (atomic) oxygen, the chemical action of which is responsible for the desired whitening/bleaching effect of whitening agents containing such peroxides. The generation of atomic oxygen is, however, highly undesirable during storage of peroxide-based teeth whitening agents because it reduces the potency of the bleaching formulation in that some of the peroxide decomposes prior to the time of use. Thus, in their commercial form, whitening agents are formulated and/or stored in a manner designed to prevent or inhibit premature peroxide decomposition. Contact with certain foreign objects, especially materials having highly developed surface areas, exposure to certain chemicals, and the presence of an elevated pH accelerate the decomposition process of said peroxides and the liberation of radical oxygen. Such conditions should be avoided.

Stability of teeth whitening formulations, however, is in direct conflict with the purpose and objective of their applications, in that the best possible whitening effect in the shortest possible time of contact with the tooth surface is achieved when the composition, when placed in contact with the teeth, is able to generate radical oxygen at a high rate and in a reasonably short time. The compromise between the objectives of high efficacy and commercially feasible storage stability is difficult to achieve for both carbamide peroxide and hydrogen peroxide-based formulations. Consequently, teeth whitening formulations known in the art typically require multiple applications stretching over a period of weeks or even months, with each recommended application time usually being from two to eight hours.

The concentrations of peroxides in commercially available teeth bleaching formulations vary greatly, generally depending on factors such as recommended time of a single application, frequency and technique of application.

To provide prolonged contact with whitening formulation with teeth while minimizing contact with mucosa which may cause irritation, the whitening material is usually placed on fabricated trays, preferably those which have been custom fit in a dentist's office to precisely conform to the patient's anatomy. The use of more highly concentrated H₂O₂ formulations, which are generally faster acting, calls for special measures to protect the mucosa from contact with such inherently irritating compositions. In such cases, rubber dams or curable tissue coatings are routinely used to protect soft tissues. Such formulations are also generally less stable during storage and the high concentration of peroxide may be aggressive against other components in the whitener formula.

Attempts have been made to accelerate the teeth bleaching processes without increasing the concentration of the peroxide by using heat-generating devices such as high intensity light emitting instruments or lasers. Because of the cost of necessary equipment and the greatly increased risk of tissue damage associated with these techniques, they are designed for use exclusively by a trained dental professional. The most effective of these techniques appear to be those using lasers, but such techniques also carry the highest risk of inflicting damage on the teeth and/or soft tissue. Furthermore, the treatment cost is considerably higher than when conventional methods are used.

### SUMMARY OF THE INVENTION

In preferred embodiments, there is provided a one or multi-component teeth whitening system wherein the one component or the first of the multiple components comprises: about 1%-12% hydrogen peroxide; about 5%-22% carbamide peroxide; about 4%-50% water; and about 25%-75% organic water-miscible solvent. In multi-component systems, a second component preferably comprises a solution or suspension of alkaline or alkaline earth oxides, peroxides, hydroxides or salts. In preferred embodiments, the organic solvent comprises a hydroxyl group-containing compound, preferably glycerin, propylene glycol, ethyl alcohol or their mixtures.

Preferred embodiments preferably further comprise one or more thickening agents, including silica; polyacrylic acid and/or its derivatives; polyalkylene oxides such as polyethylene oxide, polypropylene oxide and/or their copolymers; and derivatives of cellulose, including alkyl cellulose, hydroxyalkyl cellulose, carboxyalkyl cellulose and/or their derivatives; and/or one or more stabilizing agents, preferably those selected from the group consisting of sodium trisilicate, 1-hydroxyethylidene-1,1phosphonic acid, sodium aluminate, diethylenetriamine penta (methylene phosphonic) acid, sodium stannate, aluminum phosphate, sodium pyrophosphate, 8-hydroxyquinoline, amino trimethylenephosphonic acid, diethylenetriamine penta acetate pentasodium salt, ethylenediamine tetra acetate tetrasodium salt, magnesium sulfate, diethanol glycine, sodium tripolyphosphate, and trisodium nitrilotriacetate.

In preferred teeth whitening systems, the pH of the one or first component is about 3.5-6.5, and the pH of the second component is about 8-11.5. In systems comprising two components, a blend of the first and second components preferably has a pH of about 6-11, and the first and second components are blended in volumetric proportions ranging from 5:1 to 1:5.

In accordance with another embodiment, there is provided a method of whitening teeth. The method comprises applying a whitening system to one or more teeth, said whitening system comprising a first component comprising about 1%-12% hydrogen peroxide, about 5%-22% carbamide peroxide, about 4%-50% water, and about 25%-75% at least one organic water miscible solvent, and leaving the whitening composition in contact with said teeth. In one embodiment, the whitening system comprises a second component comprising a solution or suspension of at least one alkaline or alkaline earth oxides, hydroxides, peroxides or salts, and the first and second components are mixed together prior to applying.

Unless noted otherwise, percentages are by weight, and amounts given for a particular component of a formulation is the total amount of that component, which may consist of one or more individual compounds or materials.

### DETAILED DESCRIPTION OF THE PREFERRED EMBODIMENTS

Disclosed are new teeth whitening compositions comprising one or two components. In two component systems, the components are separated from one another during storage and mixed immediately before or during their application to the teeth. The single component or one of the two components of the composition comprises a mixture of hydrogen and carbamide peroxides in a solution having the consistency of a gel or a viscous liquid. Such solution may comprise about 1-12% hydrogen peroxide and about 5-22% carbamide peroxide to yield a blend. In one embodiment, a preferred blend contains active peroxide at concentrations corresponding to about 4.8-12.6% hydrogen peroxide (about 13.6-35% carbamide peroxide). Such solutions may be employed as a one component tooth whitener or as a two component system, the other component representing, preferably, a gel or paste consistency solution or suspension of alkaline or alkaline earth metal oxides, peroxides, hydroxides, or their salts with weak acids. Such formulations show distinct advantages over the materials based solely on carbamide or hydrogen peroxides, featuring greater peroxide concentration and consistency stability, faster teeth whitening action and reduced soft tissue irritation potential.

As discussed above, prior art teeth whitening formulations based on either carbamide peroxide or hydrogen peroxide alone, can have serious shortcomings with respect to their peroxide stability, stability of consistencies upon storage, efficacy or compatibility with oral tissues.

It was therefore surprising and entirely unexpected to find that a combination of these two peroxides in a medium containing water and at least one organic solvent allows one to formulate teeth whiteners having high peroxide concentrations, including but not limited to peroxide contents equivalent to concentration of hydrogen peroxide above about 12% (or carbamide peroxide of above about 33%), while featuring good peroxide stability, stability of physical characteristics and high teeth whitening efficacy while being biocompatible and adaptable to various techniques of application.

As discussed above, prior art high efficacy teeth whitening formulations tend to have one or more shortcomings, rendering them troublesome or undesirable. Many of the difficulties arise from the inherent conflict between the requirements of shelf life stability and the understandable demand for high efficacy of the product. Part of this conflict arises from the fact that storage stability requirements impose a need for maintaining low pH of commercial teeth whitening formulations, especially those based on hydrogen peroxide. This can be objectionable from the point of view of the potentially damaging effect on teeth and mucosa and the slow generation of radical (atomic) oxygen in the oral environment, which impairs the speed and efficacy of the teeth bleaching process. Some of the more recent fast acting two component teeth bleaching formulations, containing either hydrogen peroxide or carbamide peroxide as the active ingredient, require the use of highly alkaline materials to bring the pH of the final mixture above 11. Such highly alkaline materials could cause irritation of mucosa, especially in cases of scarified or inflamed tissue.

Preferred embodiments disclosed herein overcome some or all of the shortcomings of the prior art formulations as discussed above. In preferred embodiments, fast acting teeth whitening compositions are provided that minimize tissue desiccation, are substantially less sensitive to ambient temperatures, and are not compromised by unduly short shelf lives.

The new tooth whitening systems comprise a single component or two components where such two components are separated from each other during storage. The single component or the first of the two components comprises about 1-12% hydrogen peroxide in a free form, including 2%, 3%, 4%, 5%, 6%, 7%, 8%, 9%, 10%, 11% and ranges encompassing two or more of such amounts and about 5-22% hydrogen peroxide in a form of an adduct with urea (carbamide peroxide), including 6%, 8%, 10%, 12%, 14%, 16%, 18%, and 20% and ranges encompassing two or more of such amounts. Other ingredients include organic solvents and water. Preferred organic solvents include water-miscible solvents including those having one or more hydroxyl-groups, including but not limited to glycerin, glycols including propylene glycol, and alcohols including ethyl alcohol, and mixtures thereof. The total amount of organic solvent is preferably about 25% to 75%, including about 30%, 35%, 40%, 45%, 50%, 55%, 60%, 65%, and 70% and ranges encompassing two or more of such amounts. Water is preferably present at a concentration of about 4%-50%, including about 5%, 10%, 15%, 20%, 25%, 30%, 35%, 40%, and 45% and ranges encompassing two or more of such amounts. In preferred embodiments, the pH of the single or first component is preferably about 3.5-6.5, including about pH 4, 4.5, 5, 5.5, and 6 and ranges encompassing two or more of such values, and the preferred consistency is that of a gel or paste.

In some embodiments, one or more of the following optional ingredients are included: thickening/gelling agents or fillers, peroxide stabilizers, flavors, coloring agents and fluoridizing agents. Preferred thickening agents include silica, polyacrylic acid and its derivatives, polyalkylene oxides including polyethylene and polypropylene oxide, polyglycols, natural and synthetic gums and cellulose derivatives, including alkyl cellulose, hydroxyalkyl cellulose, carboxyalkyl cellulose and their derivatives. Preferred peroxide stabilizers include sodium pyrophosphate, 1-hydroxyethylidene-1,1-phosphonic acid and diethylenetriamine penta (methylene phosphonic acid). In preferred embodiments, thickening agents are optionally provided at concentrations of about 2% to 20%, including 5%, 10%, 15% and ranges encompassing two or more of such amounts, peroxide stabilizers are optionally provided at concentrations of about 0.1 to 2%, and/or flavorings and/or colorants are optionally provided at concentrations of about 0.01% to about 1%. The foregoing amounts may comprise one or more specific compounds or materials.

If the teeth whitening system comprises two components, the second component comprises a solution of suspension of peroxides, oxides, hydroxides and/or salts of elements belonging to the first or second group of the periodic table, examples of which include, but are not limited to, sodium and potassium hydroxides, carbonates, bicarbonates, acetates and citrates, and calcium, magnesium, strontium and zinc oxides, hydroxides, peroxides, acetates, carbonates, silicates and citrates. The component may include one or more of these materials. These materials are present in a quantity of about 2-10%, including 4%, 6%, 8% and ranges encompassing two or more of these values. These ingredients are dissolved or suspended in a liquid medium, preferably comprising one or more of water, glycerin, propylene glycol, other water-miscible organic solvents at concentrations of about 75% to 90%.

Optional ingredients of the second component include inorganic or organic fillers, thickening/gelling agents, natural or artificial sweeteners, coloring, flavoring agents and fluoridizing agents. Preferred sweeteners include xylitol, aspartame, saccharine, and fructose. Preferred fillers include silica, calcium carbonate, calcium silicate, polyethylene and polypropylene. Preferred thickening/gelling agents include polyacrylic acid and its derivatives, polyalkylene oxides, cellulose derivatives, xanthan gum and other natural and synthetic gums. Preferred fluoridizing agents include sodium fluoride, stannous fluoride, sodium monofluorphosphate and zinc and sodium hexafluorosilicates. In preferred embodiments, thickening agents are optionally provided at concentrations of about 1-5%, fillers are optionally provided at concentrations of about 1-5%, and flavorings and/or colorants are optionally provided at concentrations of about 0.01-1 %. The pH of the second component is preferably in the range of about 8 -11.5, more preferably, about 9.5-10.5, including pH 8.5, 9, 10, and 11. The consistency of the second component is preferably a gel or soft paste. In one embodiment, the two components (after mixing in a 1:1 proportion by volume) preferably yield a blend having pH of about 6-11, more preferably about 8.5-10.5, including pH 6.5, 7, 7.5, 8, 9, 9.5, and 10. The components may also be mixed in volumetric proportions ranging from 5:1 to 1:5.

Preferred formulations are stable with regard to peroxide concentration preferably for at least 60 days at 37° C. and/or for at least 90 days at room temperature (about 18-25° C.).

Whitening of the teeth is effected by applying a composition as described hereinabove to the teeth and leaving it in contact with the teeth. The contact period is usually several minutes, but may be longer, during which time the teeth will become whiter or less discolored. In embodiments having a two-component whitening system, the whitener is preferably used immediately after mixing.

### EXAMPLES OF PREFERRED FORMULATIONS

For better illustration, selected embodiments are illustrated in the Examples below. All percentages below and elsewhere herein are by weight, unless noted otherwise.

### Example 1

The teeth whitening single component system comprised:

| | |
|---|---|
| Water | 34.5% |
| Glycerin | 34% |
| Hydrogen peroxide | 11% |
| Carbamide peroxide | 5% |
| Polyethylene oxide | 13% |
| Phosphoric acid | 0.3% |
| Polyacrylic acid | 0.3% |
| 1-hydroxyethylidene-1,1-phosphonic acid | 0.2% |
| Sodium pyrophosphate | 1.5% |
| Peppermint oil | 0.2% |

The pH of the material was 5.9 and it had a consistency of a flowable gel. The peroxide contents did not change within 0.2% error margin during 60 days exposure to 37° C. or during storage at 20° C. for periods exceeding 90 days. The consistency of the blend has remained virtually stable when stored at temperatures not exceeding 20° C. for periods exceeding 90 days.

### Example 2

The teeth whitening single component system comprised:

| | |
|---|---|
| Water | 36.5% |
| Propylene glycol | 27.3% |
| Copolymer of ethylene and propylene oxides | 15.5% |
| Carbamide peroxide | 15% |
| Hydrogen peroxide | 3% |
| Sodium pyrophosphate | 1.0% |
| Polyacrylic acid | 1.55% |
| 1-hydroxyethylidene-1,1-phosphonic acid | 0.15% |

The pH of the material was 5.4. The peroxide contents did not change within 0.2% error margin during 60 days exposure to 37° C. The consistency of the material has remained virtually stable during the same period or during 90 days storage at ambient temperatures of 18-25° C.

### Example 3

The teeth whitening two-component system comprised:

Part 1:

| | |
|---|---|
| Water | 42% |
| Glycerin | 30% |
| Polyethylene oxide | 12.9% |
| Carbamide peroxide | 9% |
| Hydrogen peroxide | 3.3% |
| Sodium pyrophosphate | 1.6% |
| Phosphoric acid | 1.2% |

Part 2:

| | |
|---|---|
| Glycerin | 85.90% |
| Sodium carbonate | 3.50% |
| Sodium bicarbonate | 2.20% |
| Xylitol | 3.50% |
| Aluminum oxide | 2.20% |
| Polyacrylic acid | 1.70% |
| Peppermint oil | 0.50% |
| FD&C Blue #1 | 0.45% |
| FD&C Blue #2 | 0.05% |

The pH of the first component was 5.5. The pH of the second component was 9.6. The pH of the blend of both components at volumetrically 1:1 ratio was 8.8. The blue coloration of the blend has disappeared after 9 minutes of exposure to 37° C. The consistency of both parts has remained virtually unchanged during 90 days storage at ambient temperatures of 18-25° C. During the same period the peroxide contents in the first component has remained stable within 0.2% error margin.

### Example 4

The teeth whitening two-component system comprised:

Part 1:

| | |
|---|---|
| Water | 41.2% |
| Glycerin | 26.5% |
| Polyethylene oxide | 14.1% |
| Carbamide peroxide | 10% |
| Hydrogen peroxide | 7.2% |
| Sodium pyrophosphate | 0.6% |
| 1-hydroxyethylidene-1,1-phosphonic acid | 0.1% |
| Polyacrylic acid | 0.3% |

Part 2:

| | |
|---|---|
| Water | 63% |
| Glycerin | 20% |
| Polyether | 3.2% |
| Xylitol | 4.5% |
| Silica | 3% |
| Na-carboxymethyl cellulose | 2.6% |
| Sodium bicarbonate | 2% |
| Sodium carbonate | 1% |
| Calcium hydroxide | 0.2% |
| Peppermint oil | 0.5% |

The pH of the first component was 5.5. The pH of the second component was 10.1. The pH of the blend of both components at volumetrically 1:1 ratio was 9.5. The blue coloration of the blend has disappeared after 8 minutes of exposure to 37° C. The consistency of both pastes has remained virtually unchanged during 60 days storage at ambient temperatures of 18-25° C. During the same period the peroxide contents in the first component has remained stable within 0.2% error margin.

### Example 5

The teeth whitening single component system comprised:

| | |
|---|---|
| Propylene glycol | 72.6% |
| Water | 4% |
| Hydrogen peroxide | 2% |
| Carbamide peroxide | 16.7% |
| Polyacrylic acid | 1.1% |
| Polyethylene oxide | 3.2% |
| Diethylenetriamine penta (methylene | 0.2% |
| phosphonic) acid | |
| Sodium hydroxide | 0.2% |

The pH of the first component was 5.25. The consistency and peroxide contents of the composition have remained virtually unchanged after 90 days exposure to ambient temperatures of 18-25° C.

### Example 6

The teeth whitening two-component system comprised:

Part 1:

| | |
|---|---|
| Propylene glycol | 72.6% |
| Water | 4% |
| Hydrogen peroxide | 2% |
| Carbamide peroxide | 16.7% |
| Polyacrylic acid | 1.1% |
| Polyethylene oxide | 3.2% |
| Diethylenetriamine penta (methylene | 0.2% |
| phosphonic) acid | |
| Sodium hydroxide | 0.2% |

Part 2:

| | |
|---|---|
| Glycerin | 20% |
| Sodium carboxymethyl cellulose | 2.8% |
| Water | 63% |
| Sodium carbonate | 1.3% |
| Sodium bicarbonate | 1.9% |
| Xylitol | 4.2% |
| Polyether | 3.2% |
| Silica | 3% |
| FD&C Blue #1 and | 0.6% |
| Peppermint oil | |

The pH of the first component was 5.25. The pH of the second component was 9.9. The pH of the blend of both components at volumetrically 1:1 ratio was 9.3. The blue coloration of the blend has disappeared after 10 minutes of exposure to 37° C. The consistency and peroxide contents remained virtually unchanged after 90 days exposure to 20° C.

### Example 7

The teeth whitening two-component system comprised:

Part 1:

| | |
|---|---|
| Water | 42.5% |
| Propylene glycol | 28.6% |
| Carbamide peroxide | 8.7% |
| Hydrogen peroxide | 6.3% |
| Polyethylene oxide | 13% |
| Polyacrylic acid | 0.3% |
| 1-hydroxyethylidene-1,1-phosphonic acid | 0.1% |
| Sodium pyrophosphate | 0.5% |

Part 2:

| | |
|---|---|
| Glycerin | 23% |
| Water | 61% |
| Sodium carboxymethyl cellulose | 1.7% |
| Xylitol | 4.2% |
| Silica | 4.6% |
| Sodium carbonate | 3% |
| Sodium bicarbonate | 2% |
| Peppermint oil | 0.49% |
| FD&C Blue #1 | 0.01% |

The pH of the first component was 5.5. The pH of the second component was 10. The pH of the blend of both components at volumetrically 1:1 ratio was 9.7. The blue coloration of the blend has disappeared after 5 minutes of exposure to 37° C. The consistency and peroxide contents have remained unchanged after 60 days exposure to 20° C.

The various whitening compositions and techniques described above provide a number of ways to carry out the invention. Of course, it is to be understood that not necessarily all objectives or advantages described may be achieved in accordance with any particular embodiment described herein. Thus, for example, those skilled in the art will recognize that compositions may be formulated in a manner that achieves or optimizes one advantage or group of advantages as taught herein without necessarily achieving other objectives or advantages as may be taught or suggested herein.

Furthermore, the skilled artisan will recognize the interchangeability of various features from different embodiments. Similarly, the various features and components discussed above, as well as other known equivalents for each such feature or component, can be mixed and matched by one of ordinary skill in this art, and/or combined with other possible ingredients to make compositions in accordance with principles described herein.

Although the invention has been disclosed in the context of certain embodiments and examples, it will be understood by those skilled in the art that the invention extends beyond the specifically disclosed embodiments to other alternative embodiments and/or uses and obvious modifications and equivalents thereof. Accordingly, the invention is not intended to be limited by the specific disclosures of preferred embodiments herein, but instead by reference to claims attached hereto.

What is claimed is:

1. A one or multi-component teeth whitening system wherein the one component or the first of the multiple components comprises:
about 1%-12% hydrogen peroxide
about 5%-22% carbamide peroxide
about 4%-50% water
about 25%-75% at least one organic water miscible solvent.

2. A teeth whitening system of claim 1 further comprising a second component comprising a solution or suspension of at least one alkaline or alkaline earth oxides, hydroxides, peroxides or salts.

3. A teeth whitening system of claim 1 wherein the organic solvent comprises a hydroxyl group-containing compound.

4. A teeth whitening system of claim 3 wherein the organic solvent comprises glycerin, propylene glycol, ethyl alcohol and/or mixtures thereof.

5. A teeth whitening system of claim 1 wherein one or both components are in a physical form of a gel or paste.

6. A teeth whitening system of claim 1 wherein the first and/or second component further comprises at least one thickening agent.

7. A teeth whitening system of claim 6 wherein the thickening agent comprises silica.

8. A teeth whitening system of claim 6 wherein the thickening agent comprises polyacrylic acid or its derivatives.

9. A teeth whitening system of claim 6 wherein a thickening agent comprises a polyalkylene oxide.

10. A teeth whitening system of claim 6 wherein the thickening agent comprises at least one derivative of cellulose, including alkyl cellulose, hydroxyalkyl cellulose, carboxyalkyl cellulose and their derivatives.

11. A teeth whitening system claim 2 wherein the second component further comprises at least one coloring agent.

12. A teeth whitening system of claim 2 wherein the first and/or second component further comprises at least one teeth fluoridizing agent selected from the group consisting of sodium fluoride, stannous fluoride, sodium monofluorophosphate, zinc hexafluorosilicate and sodium monofluorosilicate.

13. A tooth whitening system of claim 2 wherein either or both of the components further comprises flavoring agents.

14. A teeth whitening system of claim 1 further comprising one or more stabilizing agents selected from the group consisting of sodium trisilicate, 1-hydroxyethylidene-1,1 phosphonic acid, sodium aluminate, diethylenetriamine penta (methylene phosphonic) acid, sodium stannate, aluminum phosphate, sodium pyrophosphate, 8-hydroxyquinoline, amino trimethylenephosphonic acid, diethylenetriamine penta acetate pentasodium salt, ethylenediamine tetra acetate tetrasodium salt, magnesium sulfate, diethanol glycine, sodium tripolyphosphate, trisodium nitrilotriacetate.

15. A teeth whitening system of claim 1 wherein the pH of the component is about 3.5-6.5.

16. A teeth whitening system of claim 2 wherein the first component further comprises at least one stabilizing agent selected from the group consisting of sodium trisilicate, 1-hydroxyethylidene-1,1 phosphonic acid, sodium aluminate, diethylenetriamine penta (methylene phosphonic) acid, sodium stannate, aluminum phosphate, sodium pyrophosphate, 8-hydroxyquinoline, amino trimethylenephosphonic acid, diethylenetriamine penta acetate pentasodium salt, ethylenediamine tetra acetate tetrasodium salt, magnesium sulfate, diethanol glycine, sodium tripolyphosphate, trisodium nitrilotriacetate.

17. A teeth whitening system of claim 2 wherein the pH of the first component is about 3.5-6.5.

18. A teeth whitening system of claim 2 wherein the pH of the second component is about 8-11.5.

19. A teeth whitening system of claim 2 wherein the blend of the first and second components have a pH of about 6-11.

20. A teeth whitening system of claim 2 wherein the blend of the first and second components have a pH of about 8.5-10.5.

21. A teeth whitening system of claim 2 wherein the first and second components are blended in volumetric proportions ranging from 5:1 to 1:5.

22. A method of whitening teeth comprising:
applying whitening system to one or more teeth, said whitening system comprising a first component comprising about 1%-12% hydrogen peroxide, about 5%-22% carbamide peroxide, about 4%-50% water, and about 25%-75% at least one organic water miscible solvent; and
leaving the whitening composition in contact with said teeth.

23. The method of whitening teeth of claim 22, wherein the whitening system comprises a second component comprising a solution or suspension of at least one alkaline or alkaline earth oxides, hydroxides, peroxides or salts, and the first and second components are mixed together prior to applying.
